SEQUENCE LISTING (19 sequences)

SEQ ID NO: 1 - DNA, 20 bp, synthetic construct
  source 1..20: mol_type = other DNA
cttaccatcaaagaagctct

SEQ ID NO: 2 - DNA, 20 bp, synthetic construct
  source 1..20: mol_type = other DNA
ctcaccagattcccgaaggt

SEQ ID NO: 3 - DNA, 20 bp, synthetic construct
  source 1..20: mol_type = other DNA
ccttaccttgagaagtttag

SEQ ID NO: 4 - DNA, 20 bp, synthetic construct
  source 1..20: mol_type = other DNA
caggccttacctgaggcgag

SEQ ID NO: 5 - DNA, 20 bp, synthetic construct
  source 1..20: mol_type = other DNA
tgctttacctttgcagtgat

SEQ ID NO: 6 - DNA, 20 bp, synthetic construct
  source 1..20: mol_type = other DNA
cacctacctaagaaccatcc

SEQ ID NO: 7 - DNA, 20 bp, synthetic construct
  source 1..20: mol_type = other DNA
ttgacactgccaaacctatt

SEQ ID NO: 8 - DNA, 20 bp, synthetic construct
  source 1..20: mol_type = other DNA
aaatatatcaccactattgc

SEQ ID NO: 9 - DNA, 20 bp, synthetic construct
  source 1..20: mol_type = other DNA
atgacactaagtcaagttaa

SEQ ID NO: 10 - DNA, 20 bp, synthetic construct
  source 1..20: mol_type = other DNA
attcttgatctcatctattt

SEQ ID NO: 11 - DNA, 20 bp, synthetic construct
  source 1..20: mol_type = other DNA
tacaggatccagatctaact

SEQ ID NO: 12 - DNA, 20 bp, synthetic construct
  source 1..20: mol_type = other DNA
agtgactgacatcaactcca

SEQ ID NO: 13 - DNA, 20 bp, synthetic construct
  source 1..20: mol_type = other DNA
gtgactgacatcaactccaa

SEQ ID NO: 14 - DNA, 20 bp, synthetic construct
  source 1..20: mol_type = other DNA
gtgagacgttgactgagtgc

SEQ ID NO: 15 - DNA, 20 bp, synthetic construct
  source 1..20: mol_type = other DNA
tgagacgttgactgagtgct

SEQ ID NO: 16 - DNA, 20 bp, synthetic construct
  source 1..20: mol_type = other DNA
gggctgtgagacgggcctct

SEQ ID NO: 17 - DNA, 20 bp, synthetic construct
  source 1..20: mol_type = other DNA
ccgtctcacagcccagtgtg

SEQ ID NO: 18 - DNA, 20 bp, synthetic construct
  source 1..20: mol_type = other DNA
cccgctacagggcatccaga

SEQ ID NO: 19 - DNA, 20 bp, synthetic construct
  source 1..20: mol_type = other DNA
gtagacatcggtctgcttga

(19) United States
(12) Patent Application Publication
(10) Pub. No.: US 2024/0352420 A1
(43) Pub. Date: Oct. 24, 2024

(54) MULTIPLEX BASE EDITING OF PRIMARY HUMAN NATURAL KILLER CELLS

(71) Applicant: Regents of the University of Minnesota, Minneapolis, MN (US)

(72) Inventors: Minjing WANG, Minneapolis, MN (US); Branden S. MORIARITY, Minneapolis, MN (US); Beau R. WEBBER, Minneapolis, MN (US); Mitchell G. KLUESNER, Minneapolis, MN (US); Emily J. POMEROY, Minneapolis, MN (US); Bryce WICK, Minneapolis, MN (US)

(21) Appl. No.: 18/293,288

(22) PCT Filed: Jul. 29, 2022
(86) PCT No.: PCT/US2022/074331

§ 371 Date: Jan. 29, 2024

Related U.S. Application Data
(60) Provisional application No. 63/226,952, filed on Jul. 29, 2021.

(51) Int. Cl. C12N 5/0783; A61K 35/17; C12N 9/22; C12N 9/78; C12N 15/11

(52) U.S. Cl. CPC C12N 5/0646; A61K 35/17; C12N 9/22; C12N 9/78; C12N 15/111; C12N 2310/20; C12N 2510/00

(57) ABSTRACT

The present invention provides efficient methods for producing genetically modified natural killer (NK) cells using a base editor and guide RNA(s). Genetically modified NK cells produced by these methods and the use of these cells in the treatment of cancer are also provided.

### CROSS-REFERENCE TO RELATED APPLICATIONS

The present application claims priority to U.S. Provisional Patent Application No. 63/226,952 filed Jul. 29, 2021, the entire contents of which are hereby incorporated by reference.

### STATEMENT REGARDING FEDERALLY SPONSORED RESEARCH

This invention was made with government support under grant number W81XWH-19-1-0243 awarded by the Department of Defense. The government has certain rights in this invention.

### REFERENCE TO AN ELECTRONIC SEQUENCE LISTING

The contents of the electronic sequence listing (92171.00485.xml; Size: 17,618 bytes; and Date of Creation: Jul. 29, 2022) is herein incorporated by reference in its entirety.

### BACKGROUND

Natural killer (NK) cells show great potential for use in cancer immunotherapies. NK cells can kill cancer cells in an antigen-independent fashion and perform antibody-dependent cell-mediated cytotoxicity (ADCC). Further, NK cells can be used as an allogeneic product. However, the function of these cells relies on the balance of inhibitory and activating signals, which are often manipulated by cancers to avoid immune surveillance. For example, in patients with cancer, NK cell function is generally inhibited due to reduced expression of NK cell-activating receptors and upregulation of inhibitory receptors, which impairs their tumor-killing activity. Further, the tumor microenvironment significantly hinders the migration and penetration of NK cells into tumor site. One way to potentially disrupt these inhibitory interactions and to improve NK cell toxicity for immunotherapies is to genetically modify NK cells. However, NK cells are notoriously hard to edit.

Thus, there is a need in the art for improved methods for genetically modifying NK cells.

### SUMMARY

In a first aspect, the present invention provides methods for producing a genetically modified natural killer (NK) cell. The methods comprise introducing a combination of editing reagents into a NK cell. The editing reagents include: (a) a base editor protein or a plasmid or mRNA encoding the base editor; and (b) one or more guide RNAs (gRNAs) that comprise a spacer sequence that is complementary to a target sequence within the genome of the NK cell.

In a second aspect, the present invention provides genetically modified NK cells obtained according to the methods disclosed herein.

In a third aspect, the present invention provides uses of the genetically modified NK cells disclosed herein in a treatment for cancer.

### BRIEF DESCRIPTION OF THE DRAWINGS

FIG. 1 demonstrates that the use of an RNase inhibitor increases the knockout efficiency in NK cells. The AHR, CISH, and TIGIT genes were genetically modified in natural killer (NK) cells via introduction of the base editor ABE8e and a target-specific guide RNA, with or without the addition of an RNase inhibitor. Editing efficiency was evaluated using Sanger sequencing.

FIG. 2 demonstrates that ABE8e base editing can be used to genetically modify NK cells. Single guide RNAs (sgRNAs) targeting each gene of interest (i.e., AHR, CISH, KLRG1, TIGIT, PD-1, and CD16a) were individually introduced into primary NK cells along with mRNA encoding ABE8e. Panel A shows the editing efficiency at the DNA level for each individual target gene. Each data point is based on four NK cell donor replicates. Panel B shows the editing efficiency at the protein level for each individual target gene. Panel C includes flow cytometry analysis of the knockout efficiency for several of the target genes. Each data point is based on four NK cell donor replicates.

FIG. 3 demonstrates the multiplex editing strategy. Starting from 1 sgRNA and add 1 more sgRNA at a time to a maximum of 6 sgRNAs introduced at the same time.

FIG. 4 demonstrates that ABE8e base editing can be used for multiplex gene editing in NK cells. sgRNAs targeting six genes of interest were simultaneously introduced into primary NK cells along with mRNA encoding ABE8e. Panel A shows the editing efficiency at the DNA level for each individual target gene. Each data point is based on two NK cell donor replicates. Panel B shows the editing efficiency at the protein level for each individual target gene. Each data point is based on two NK cell donor replicates.

FIG. 5 demonstrates co-culture killing assay of WT and AHR KO NK populations against K562 target cells. The co-culture killing assay is performed at 1:2 effector to target ratio (ET ratio) for 4 hours, in +/−L-Kynurenine conditions. AHR KO NK cells demonstrate significantly improved cytotoxicity against target cells than WT NK cells, when co-cultured with L-Kynurenine. L-Kynurenine is an AHR agonist that inhibits WT NK cell cytotoxicity.

FIG. 6 demonstrates co-culture killing assay of WT and CISH KO NK populations against Nomo-1 and Molm-13 target cells. The co-culture killing assay is performed at 1:2 E:T ratio for up to 6 hours. CISH KO NK cells demonstrate significantly improved cytotoxicity against target cells than WT NK cells.

FIG. 7 demonstrates co-culture killing assay of WT and PD-1 KO NK populations against Raji and PD-L1-overexpressed Raji target cells. PD-L1 is a ligand of PD-1, and PD-1/PD-L1 interaction is a classic inhibitory immune checkpoint system that leads to reduced NK cell cytotoxicity. The co-culture killing assay is performed at 1:4 E:T ratio for up to 24 hours. PD-1 KO NK cells demonstrate significantly improved cytotoxicity against target cells, especially those with PD-L1 overexpression, than WT NK cells.

FIG. 8 demonstrates ADCC assay of WT and non-cleavable CD16a (ncCD16a) NK populations against Raji target cells. The ADCC assay is performed at 1:2 E:T ratio for up to 4 hours, with or without CD20 antibody (Ab) coating. ncCD16a NK cells demonstrate significantly improved cytotoxicity against antibody-coated target cells than WT NK cells.

FIG. 9 shows maps depicting the positions of mutations generated using base editing within Fas (A) and TGFbR2 (B).

FIG. 10 shows base editing activity of individual gRNAs targeting Fas or TGFβR2. Guides which are labeled with the letter ‘F’ are for Fas and ‘T’ are for TGFβR2.

FIG. 11 shows Fas ligand killing assay. Total live cells (A) and percent alive cells (B) after 24 hours of exposure to Fas ligand and His tag antibody or neither. Pulse cells are a control where the cells got electroporated but there was no RNA in the mixture. Only the three guides with over 50% average editing were used here.

FIG. 12 shows cell growth in the presence of TGFβ. Day 8 (A) and day 13 (B) fold expansion of T cells from first restimulation. This is calculated by comparing the total cells at the designated time points to the total cells plated during the first restimulation. Pulse cells were electroporated but there was no guide RNAs or ABE8e mRNA in the solution. Cells were restimulated and total alive cells (C) and percent alive cells (D) at day four post 3^{rd} restimulation are plotted for both the CD3 only conditions and the conditions which got both CD3 and CD28.

FIG. 13 shows DN TGFβR2 CAR T cell serial killing assay. Pulse cells got electroporated but with no RNA, TB cells have TRAC and B2M knocked out by gRNAs targeting splice donors, and TBbeta cells have TRAC and B2M knocked out like TB cells but also have the DN TGFβR2 as well. All three conditions were transduced with a CD19 CAR lentivirus (CAR+). (A) Killing of target cells over time in a serial killing assay (B) An exhaustion panel performed on the cells five days after the first replate.

### DETAILED DESCRIPTION

The present invention provides improved methods for producing genetically modified natural killer (NK) cells using a base editor and guide RNA(s). Genetically modified NK cells produced by these methods and the use of these cells in the treatment of cancer are also provided.

The use of a base editor in the present methods offers several advantages. For example, unlike CRISPR-Cas9-based gene editing, base editing does not involve induction of double stranded breaks, which makes these methods less error prone and reduces the risk of genotoxicity. Further, base editing provides a higher editing efficiency than CRISPR-Cas9-based gene editing.

The methods also utilize an RNase inhibitor, electroporation enhancers, and optimized electroporation settings to achieve increased gene editing efficiencies. In the Examples, the inventors show that their methods achieve an editing efficiency of about 75% up to 100% at both the DNA and protein levels in primary human NK cells. Further, they show that this method can be used to edit multiple genes simultaneously in NK cells.

To serve as target genes, the inventors selected a panel of genes that play a critical role in NK cell proliferation, persistence, and function in the context of cancer immunotherapy. These genes include intracellular regulators (AHR and CISH), inhibitory receptors (KLRG1, TIGIT, KLRC1, PDCD1, Fas and TGFBR2), and an Fc receptor that is responsible for antibody-dependent cellular cytotoxicity (CD16A). They designed multiple single guide RNAs (sgRNAs) to target each gene of interest. Thus, the inventors have used their novel gene editing method to produce NK cells with improved functions for use in immunotherapies. Such genetically modified NK cells could be used as an off-the-shelf immunotherapy for cancer treatment.

### Methods:

In a first aspect, the present invention provides methods for producing a genetically modified natural killer (NK) cell. The methods comprise introducing a combination of editing reagents into a NK cell. The editing reagents include: (a) a base editor protein or a plasmid or mRNA encoding the base editor; and (b) one or more guide RNAs (gRNAs) that comprise a spacer sequence that is complementary to a target sequence within the genome of the NK cell.

Natural killer (NK) cells are cytotoxic lymphocytes of the innate immune system that control several types of tumors and microbial infections by limiting their spread and subsequent tissue damage. In some embodiments, the NK cell used with the present invention is a mammalian cell, preferably a human cell.

In some embodiments, the NK cell used with the present invention is a primary cell. A “primary cell” is a cell that was taken directly from living tissue (e.g., biopsy material) and maintained in vitro. A primary NK cell may express CD16 and/or CD56. For example, in some embodiments, the primary NK cell is CD56+ and CD3− or is CD16+ and CD3−. In some embodiments, the NK cell is a primary cell that was collected from a patient. For example, a primary NK cell may be isolated from peripheral blood, umbilical cord cells, ascites, or a solid tumor. The primary NK cell may be freshly isolated or it may have undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated. In some embodiments, the primary NK cell is a non-clonal cell. In some embodiments, the primary NK cell is a proliferating cell. In some embodiments, the primary NK cell is an expanded cell. In some embodiments the NK cell is derived from a population of stem cells including but not limited to human induce pluripotent stem cells, or hematopoietic stem cells.

In the methods of the present invention, a base editor is used to introduce a genetic modification into the NK cell. As used herein, a “base editor” is a fusion protein that comprises a Cas nickase domain or catalytically dead Cas protein fused to a deaminase. As in CRISPR-based gene editing, base editors are targeted to a specific gene sequences using a guide RNA (gRNA). However, unlike CRISPR, base editing does not generate double-stranded DNA breaks, making it a safer alternative to Cas nuclease-based methods. Instead, base editing uses the deaminase enzyme to modify a single base without altering the bases around it. There are two classes of base editors: cytosine base editors (CBEs) and adenine base editors (ABEs). CBEs comprise a cytidine deaminase that converts cytidine to uridine within a small editing window near the protospacer adjacent motif (PAM) site. Uridine is subsequently converted to thymidine through base excision repair, creating a cytosine (C) to thymine (T) change (i.e., a guanosine to adenine change on the opposite strand). ABEs comprise an adenine deaminase, which creates an adenine (A) to guanosine (G) change. When a CBE is utilized, to prevent cells from repairing the modified base and encourage the cell to use the edited strand as a template for mismatch repair, a uracil DNA glycosylase inhibitor (UGI) is used to block base excision repair. In some embodiments, a UGI domain is included as part of the base editor fusion protein. In other embodiments, the UGI domain is provided to the cell as a separate component. Researchers have developed third and fourth generation base editors with improved efficiency. For example, the third generation CBE base editor BE3 (i.e., base editor 3) uses a Cas9 nickase to nick the unmodified DNA strand so that it appears “newly synthesized” to the cell, forcing the cell to repair the DNA using the deaminated strand as a template, whereas fourth generation base editors systems (i.e., base editor 4 (BE4)) employ two copies of base excision repair inhibitor UGI. In some embodiments, a BE3 or BE4 cytosine base editor is used in the methods of the present invention. In other embodiments, a CBE comprising a different deaminase, such as hA3A-BE4, hA3G-BE4, evoFERNY-BE4, or evoCDA-BE4, is used. In other embodiments, an ABE base editor, such as ABE6.3, ABE7.10, ABE8e, or ABE8.20 is used. In some embodiments, the base editor enzymes are mutated or modified to confer a desired functionality such as reduced guide-independent off-target editing, reduced guide-dependent off-target editing, an altered editing window, an altered editing context preference, an altered target site specificity, or more precise target editing. The base editor may be those known or developed in the art using other enzymatic variants. In certain embodiments, Cas9 variants are employed with expanded PAM recognitions, including but not limited to SaCas9-KKH, SpCas9-VQR, SpCas9-VRER, SpCas9-NG, SpRY-Cas9, SpCas9-NRCH, and xCas9. In certain embodiments, different Cas9 orthologs or paralogs are used including but not limited to SaCas9, FnCas9, NmeCas9, AsCas12a, FnCas12a. The base editor can be provided to the NK cell in the form of a base editor protein or in the form of a plasmid or mRNA encoding the base editor protein.

A “guide RNA (gRNA)” is an RNA molecule that targets an enzyme to a specific genomic sequence via complementary base pairing. The gRNAs used with the present invention comprise a sequence that is complementary to a target sequence within the genome of the NK cell. The complementary portion of a gRNA comprises at least 10 contiguous nucleotides, and often comprises 17-23 contiguous nucleotides that are complementary to the target sequence. The complementary portion of the gRNA may be partially or wholly complementary to the target sequence. In some embodiments, the gRNA is from 20 to 120 bases in length, or more. In certain embodiments, the gRNA can be from 20 to 60 bases, 20 to 50 bases, 30 to 50 bases, or 39 to 46 bases in length. Various online tools and software environments can be used to design an appropriate gRNA for a particular application. In some embodiments, the gRNA is a chemically modified gRNA. For example, the gRNA may be chemically modified to decrease a cell's ability to degrade the gRNA. Suitable chemically modified gRNAs may include one or more of the following modifications: 2′-fluoro (2′-F), 2′-O-methyl (2′-O-Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the gRNA is composed of two molecules that base pair to form a functional gRNA: one comprising the region that binds to the base editor and one comprising a targeting sequence that binds to the target site. Alternatively, the gRNA may be a single molecule comprising both of these components, i.e., a single guide RNA (sgRNA).

The methods of the present invention involve introducing or transfecting the editing reagents (i.e., nucleic acids and, in some embodiments, protein) into an NK cell. As used herein, the term “transfection” refers to any process by which nucleic acids and/or proteins are introduced into eukaryotic cells. Any suitable method of introducing a nucleic acid or protein into a cell may be used including, without limitation, those that utilize electroporation, microinjection, viral delivery, exosomes, liposomes, nanoparticles, biolistics, jet injection, hydrodynamic injection, ultrasound, magnetic field-mediated gene transfer, electric pulse-mediated gene transfer, incubation with an endosomolytic agent, or cell-penetrating peptides.

In some embodiments, introduction of the editing reagents is accomplished by electroporation. “Electroporation” is a physical transfection method that uses an electrical pulse to create temporary pores in cell membranes through which substances like nucleic acids and proteins can pass into cells. Methods for performing electroporation are well known in the art. In the Examples, the inventors used the Neon™ Transfection System (Thermo Fisher Scientific Inc.) to perform electroporation. The Neon™ Transfection System utilizes a proprietary biologically compatible pipette tip chamber to generate a more uniform electric field, which significantly increases transfection efficiency and cell viability. Thus, in some embodiments, introduction of editing reagents is accomplished using the Neon™ Transfection System.

The inventors have found that they can increase the gene editing efficiency achieved by their method by adding an electroporation enhancer to the editing reagents prior to electroporation. As used herein, an “electroporation enhancer” is any reagent that increases the percentage of cells that are successfully transfected using a given electroporation method relative to the percentage of cells that are successfully transfected in the absence of the electroporation enhancer. Increase in transfection efficiency results in improved editing efficiency. In some embodiments, the electroporation enhancer is carrier DNA, i.e., unrelated DNA that merely serves to increase the nucleic acid concentration in the culture. In the Examples, the inventors used the Alt-R® Cas9 Electroporation Enhancer (IDT) to increase electroporation efficiency and thereby increase gene editing efficiency. The Alt-RR Cas9 Electroporation Enhancer is a single-stranded carrier DNA and that was computationally designed to be non-homologous to human, mouse, and rat genomes. This electroporation enhancer was optimized to work with the Neon™ Transfection System (Thermo Fisher Scientific Inc.). Thus, in some embodiments, Alt-R® Cas9 Electroporation Enhancer is added to the editing reagents prior to electroporation.

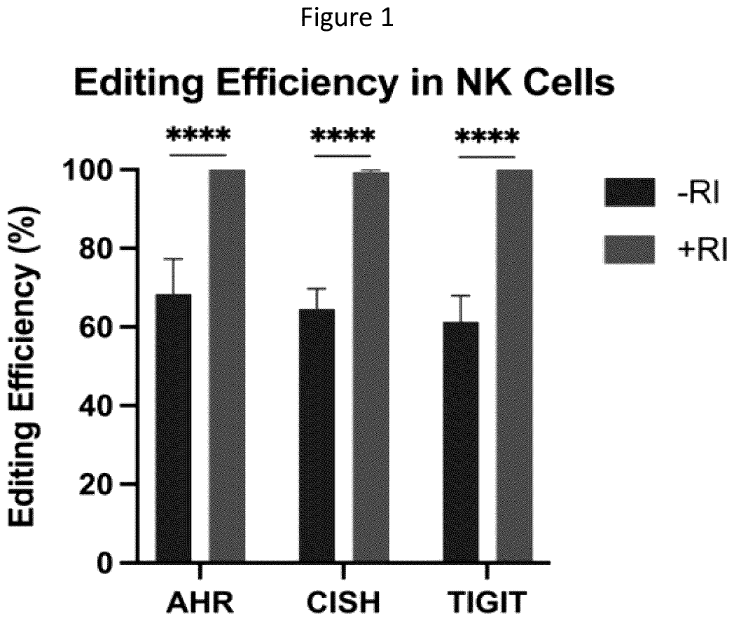

Additionally, the inventors have discovered that they can increase the gene editing efficiency achieved by their method if they use an RNase inhibitor (see FIG. 1). “RNase inhibitors” are reagents that inhibit or inactivate certain ribonuclease enzymes (i.e., RNases; enzymes that degrade RNA). In the Examples, the inventors utilized Protector RNase Inhibitor (Millipore Sigma), which is a 50 kD protein that inhibits the enzymatic activity of RNases by noncovalently binding to their active site. Thus, in some embodiments, an RNase inhibitor is added to the editing reagents before they are introduced into the NK cell to protect the RNA molecule(s) from degradation.

Electroporation may be performed under various settings. For example, in some embodiments, electroporation may include exposing the NK cell to at least 1700 volts, at least 1750 volts, at least 1800 volts, at least 1850 volts, at least 1900 volts, at least 1950 volts, at least 2000 volts, at least 2050 volts, at least 2100 volts, or at least 2150 volts. In some embodiments, electroporation may include exposing the NK cell to up to 1850 volts, up to 1900 volts, up to 1950 volts, up to 2000 volts, up to 2050 volts, up to 2100 volts, up to 2150 volts, up to 2200 volts, or up to 2250 volts. For example, in some embodiments, a stimulated NK cell is exposed to between 1750 and 1950 volts. In other embodiments, an unstimulated NK cell is exposed to between 2100 and 2200 volts. In some embodiments, electroporation may include exposing an NK cell to multiple pulses of energy. For example, electroporation may include exposing the NK cell to at least 1 energy pulse, at least 2 energy pulses, at least 3 energy pulses, at least 4 energy pulses, or at least 5 energy pulses. In some embodiments, the NK cell may be exposed to up to 2 energy pulses, up to 3 energy pulses, up to 4 energy pulses, up to 5 energy pulses, up to 6 energy pulses, or up to 10 energy pulses. The NK cell may be exposed to an energy pulse or multiple pulses of any width (i.e., length of time). For example, a pulse may last at least 2 milliseconds, at least 3 milliseconds, at least 4 milliseconds, at least 5 milliseconds, at least 7 milliseconds, at least 9 milliseconds, at least 10 milliseconds, at least 20 milliseconds, at least 30 milliseconds, or at least 40 milliseconds. In some embodiments, at pulse may last up to 8 milliseconds, up to 10 milliseconds, up to 12 milliseconds, up to 15 milliseconds, up to 20 milliseconds, up to 30 milliseconds, up to 40 milliseconds, or up to 50 milliseconds. Notably, while any pulse width between 1-100 milliseconds may be used with the Neon™ Transfection System (Thermo Fisher Scientific Inc.), the pulse width used with this system is preferably between 10-40 milliseconds according to manufacturer instructions.

While the Neon™ Transfection System (Thermo Fisher Scientific Inc.) provides a database of validated electroporation settings for commonly used cell lines (www.thermofisher.com/us/en/home/life-science/cell-culture/transfection/neon-transfection-system/neon-transfection-system-cell-line-data.html), this database provides no guidance for the electroporation of NK cells. Accordingly, the inventors have optimized the electroporation settings that they use to base edit NK cells with this system. Advantageously, they found that the electroporation settings disclosed in the present Examples (i.e., pulse voltage: 1825 volts, pulse number: 2, pulse width: 10 milliseconds) produced higher base editing efficiencies than the settings they had previously used to edit NK cells using Cas9 (i.e., pulse voltage: 1850 volts, pulse number: 2, pulse width: 10 milliseconds; see, e.g., US Patent Application Publication No. 2020/0208111). Thus, in some embodiments, the electroporation comprises exposing the NK cell to 1825 volts and at least 2 energy pulses with a pulse width of 10 milliseconds.

At the time of transfection, the NK cell may be unstimulated (i.e., resting) or stimulated (i.e., subjected to an activation or proliferation step). In some embodiments, the NK cell is stimulated after transfection. For example, the NK cell may be stimulated beginning immediately after, one day after, two days after, three days after, four days after, five days after, six days after, seven days after, eight days after, nine days after, and/or 10 days after transfection. An NK cell may be stimulated using any suitable method and for any suitable length of time. Suitable methods to stimulating an NK cell include, for example, exposing the cell to a stimulating agent such as phorbol-12-myristate-13-acetate (PMA) or a cytokine (e.g., IL-21, IL-2, IL-12, IL-15, type I interferons, etc.). The stimulating agent may be soluble or bound to a surface. For example, the stimulating agent may be bound to the surface of a tissue culture flask or to an artificial antigen presenting cell (e.g., a bead coated with antibodies against NK cell surface proteins). The NK cell may be stimulated for hours, for days, or for weeks. For example, the NK cell may be stimulated for up to 1 day, up to 2 days, up to 3 days, up to 4 days, up to 5 days, up to 6 days, up to 7 days, up to 8 days, or up to 9 days, up to 2 weeks, up to 3 weeks, and so forth. In some embodiments, the NK cell is stimulated via treatment with a commercially available kit, such at the CellXVivo Human NK Cell Expansion Kit (R&D Systems) or the Human NK Cell Expansion Activator Kit (Miltenyi Biotech). In some embodiments, the NK cell is stimulated via co-culture with feeder cells. For instance, the inventors stimulated NK cells for use in their method by incubating the cells with K562-mbIL21-41BBL feeder cells for about 7 days.

An NK cell is “genetically modified” if its genome has been altered using genetic engineering techniques. A genetically modified NK cell contains at least one modification relative to a non-genetically-modified (e.g., wild-type) NK cell. As used herein, the terms “genetic modification” and “genetic engineering” refers to a process of artificially introducing a modification into a gene. Base editors can be used to introduce several types of modifications, including missense mutations, gene knock-outs, and gene knock-ins. In a gene knock-out, the transcription or translation of the target gene is disrupted, for example, via deletion of coding sequence, mutation of a start codon, introduction of a premature stop codon, and/or disruption of intron/exon splice sites. Gene knock-outs can also be achieved by altering the regulatory elements (e.g., promoter or enhancer) of one or more endogenous genes to prevent transcription or translation of the target gene. Gene knock-ins are used to introduce a DNA sequence and are achieved by supplying a DNA donor template from which the genome can be repaired. For example, gene knock-ins can be used to introduce DNA encoding a wild-type or mutant form of an endogenous or exogenous protein or DNA encoding an RNA molecule. Gene knock-ins may also be used to add a heterologous sequence to an endogenous gene, such as a sequence encoding a marker protein (e.g., GFP) or a chimeric antigen receptor. Thus, a genetic modification may modulate a gene in several ways, including by increasing/decreasing its expression, increasing/decreasing its activity, or introducing a new function. The methods of the present invention may be used to mutate, knock-in, and/or knock-out one or more target genes simultaneously.

In Example 1, the inventors generated mutations that abolish the expression of (i.e., knock-out) the genes aryl hydrocarbon receptor (AHR), cytokine-inducible SH2-containing protein (CISH), killer cell lectin-like receptor subfamily G member 1 (KLRG1), T cell immunoreceptor with Ig and ITIM domains (TIGIT), killer cell lectin-like receptor subfamily C member 1 (KLRC1), programmed cell death protein 1 (PDCD1), and CD16A. Specifically, to abolish expression of AHR, CISH, KLRG1, TIGIT, KLRC1, and PDCD1, the inventors introduced a point mutation that disrupted a splice donor site. To create a noncleavable form of CD16A, they introduced a missense mutation. This mutation avoids CD16A shedding and improves NK cell cytotoxicity during ADCC. Thus, in some embodiments, the method generates a mutation in the target gene that abolishes its expression or improves its function in the NK cell.

The methods of the present invention are used to generate a genetically modified NK cell in which a target sequence within a target gene is modified. As used herein, the term “target sequence” refers to the portion of the target gene to which the gRNA hybridizes via complementary base pairing. Base editing requires the presence of a protospacer adjacent motif (PAM). For example, the base editor ABE8e requires a PAM (i.e., NGG) about 12-17 bases away from the desired editing site. Thus, the target sequence should be located upstream of a PAM. Examples of PAM sequence are known in the art (see, e.g., Shah et al., RNA Biology 10 (5): 891-899, 2013).

The “target gene” may be any gene of interest found within the genome of an NK cell. Preferably, the target gene involved in the function, activation, and/or survival of NK cells. Suitable target genes include, for example, genes encoding NK activating receptors, NK inhibitory receptors, adaptor molecules, downstream signaling molecules, components of a cytotoxic granule, cytokines, chemokines, cytokine receptors, and chemokine receptors. In some embodiments, modification of the target gene confers one or more of the following properties to the NK cell: increased stimulation-induced cytokine production, increased capacity to kill cancer cells, increased survival, or increased capacity to expand relative to an unmodified NK cell.

The inventors have designed single guide RNAs (sgRNAs) that are complementary to target sequences in the following target genes: aryl hydrocarbon receptor (AHR), cytokine-inducible SH2-containing protein (CISH), killer cell lectin-like receptor subfamily G member 1 (KLRG1), T cell immunoreceptor with Ig and ITIM domains (TIGIT), killer cell lectin-like receptor subfamily C member 1 (KLRC1), programmed cell death protein 1 (PDCD1), CD16A, Fas, and transforming growth factor beta receptor 2 (TGFBR2). The sequences of these sgRNAs are provided in Table 1 and Table 2. Thus, in some embodiments, the target sequence is within a target gene selected from the above listed genes, and, in some embodiments, at least one gRNA comprises a sequence selected from the sequences provided in Table 1 and Table 2.

In Example 2, the inventors describe the generation of NK cells with modifications in two genes: Fas and transforming growth factor (TGF) beta receptor 2 (TGFBR2). These genes encode receptors that bind to the ligands FasL and TGF beta 1 (TGFB1), respectively, which are both known to be upregulated in cancer. FasL directly induces apoptosis in NK cells, whereas TGF-β is an immunosuppressive cytokine. Thus, cancer cells that express these ligands create a hostile microenvironment that shields the tumor from immunosurveillance by NK cells. Accordingly, the inventors have designed gRNAs to generate dominant negative mutations in the cognate receptors of these ligands in NK cells. As used herein, a “dominant negative mutation” is a mutation that generates a mutant gene product that adversely affects the normal, wild-type gene product within the same cell. Dominant negative mutations in Fas and TGFbR2 are expected to improve the performance of NK cells by reducing the inhibitory effects that FasL and TGFB1 have on these cells, thereby allowing them to kill cancer cells in the hostile immunosuppressive microenvironment created by FasL- and TGFB1-expressing cancer cells. Thus, in some embodiments, the method generates a dominant negative mutation in the NK cell.

Specifically, the inventors have designed gRNAs that disrupt splicing of Fas (i.e., in exon 6) or create substitution mutations in Fas that are known to be associated with autoimmune lymphoproliferative syndrome (ALPS) or that mimic mutations seen in ALPS (i.e., T28A, Y232S, T241A, I262T). ALPS is a rare inherited disorder in which the body cannot properly regulate the number of lymphocytes such that these cells accumulate in the lymph nodes, liver, and spleen and can lead to enlargement of these organs. Deleterious heterozygous mutations in the FAS gene are the most common cause of this condition. In fact, 75% of all ALPS cases and 100% of classical ALPS cases involve mutations in Fas. Thus, the mutations found in this disease are expected to disrupt Fas signaling. For use in their method, the inventors selected ALPS-mimicking Fas mutations that can be made using the base editor ABE8e. Accordingly, in some embodiments, the target gene is Fas and the genetically modified NK cell comprises a mutation in Fas selected from T28A, Y232S, T241A, and I262T.

Similarly, the inventors have designed gRNAs that disrupt splicing in TGFbR2 (i.e., in exon 8) or create substitution mutations in TGFbR2 that mimic those seen in Loeys-Dietz syndrome type II (i.e., T516A, L529P, T530A, V447). Loeys-Dietz syndrome (LDS) is a genetic disorder that affects the connective tissue in the body. LDS is caused by a genetic mutation in one of five genes that encode proteins involved in the transforming growth factor-beta signaling pathway. Specifically, LDS type I is caused by a mutation in transforming growth factor beta-receptor 1 (TGFbR1) and LDS type II is caused by a mutation in transforming growth factor beta-receptor 2 (TGFbR2), whereas LDS types 3-5 are caused by mutations in the genes mothers against decapentaplegic homolog (SMAD-3), transforming growth factor beta-2 ligand (TGFb2), and transforming growth factor beta-3 ligand (TGFb3), respectively. Thus, the mutations found in LDS type II are expected to disrupt TGFbR2 signaling. For use in their method, the inventors selected LDS type II-mimicking TGFbR2 mutations that could be made using the base editor ABE8e. Accordingly, in some embodiments, the target gene is TGFBR2 and the genetically modified NK cell comprises a mutation in TGFBR2 selected from T516A, L529P, T530A, and V447.

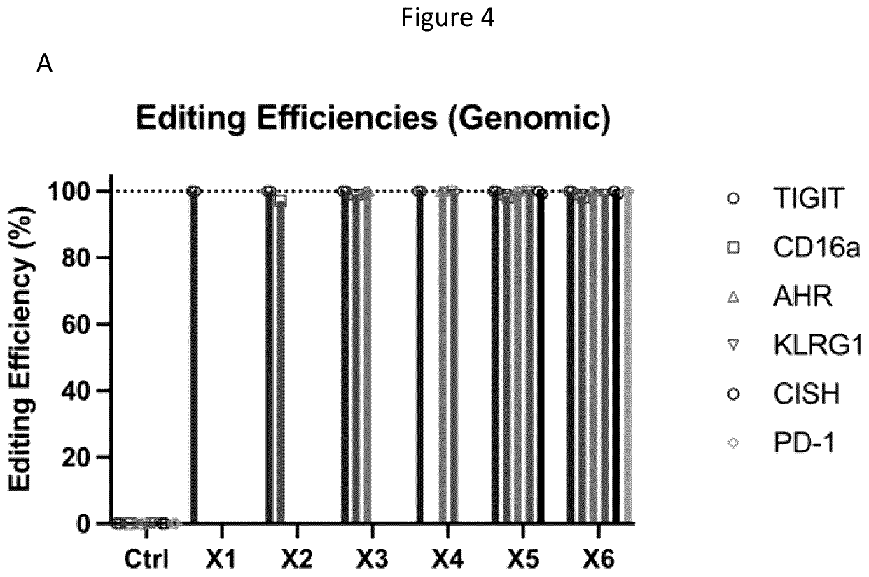

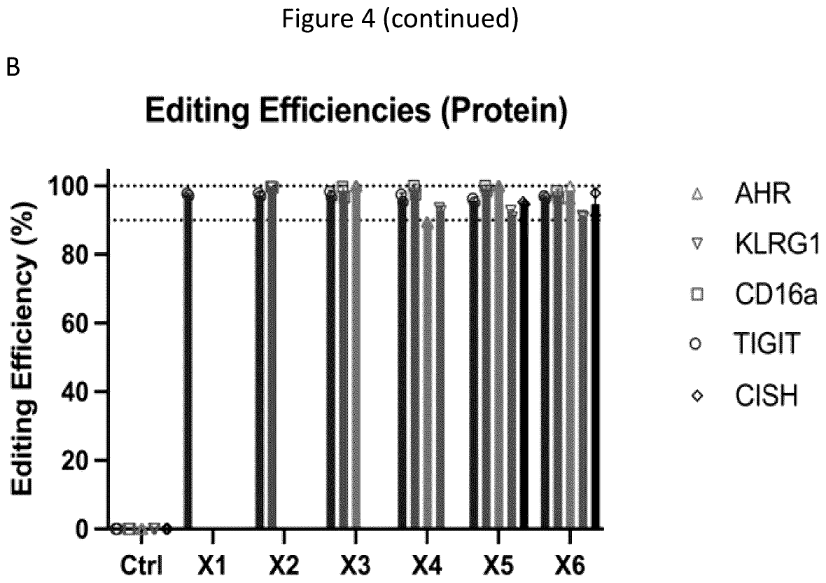

In Example 1, the inventors demonstrate that their high efficiency base editing method can be used for multiplex editing (see FIG. 4). Thus, in some embodiments, the methods are used to generate a genetically modified NK cell in which at least two target sequences have been modified. In these embodiments, at least two gRNAs that are each designed to target a different sequence within the genome of the NK cell are introduced into the NK cell. In some cases, one or more genes are disrupted and one or more genes is knocked-in using a donor DNA template for insertion.

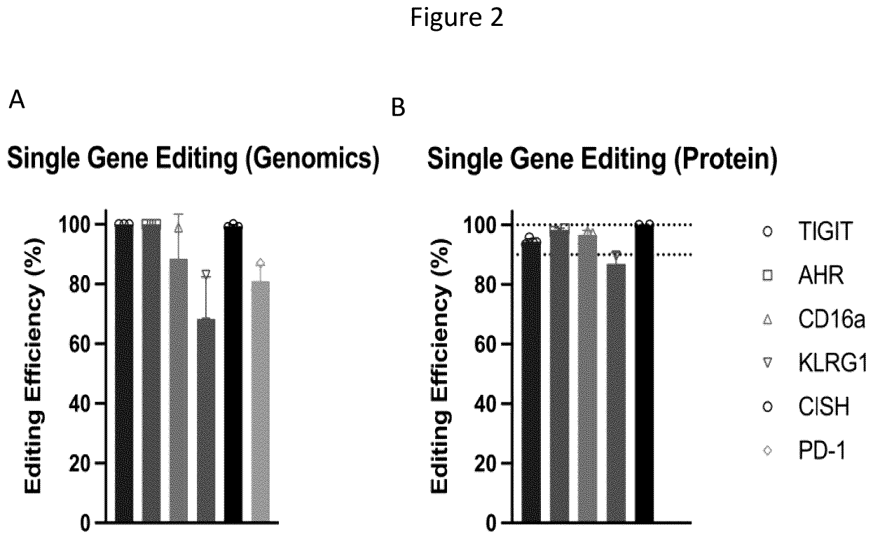

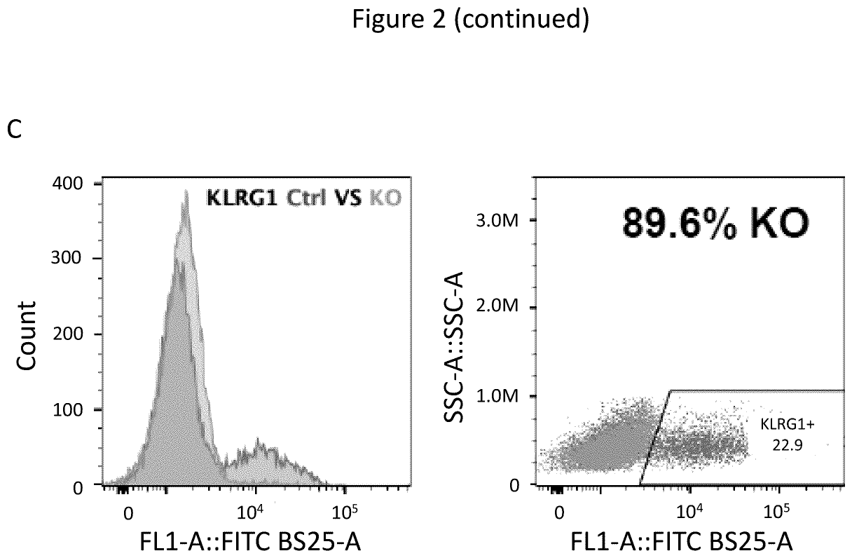

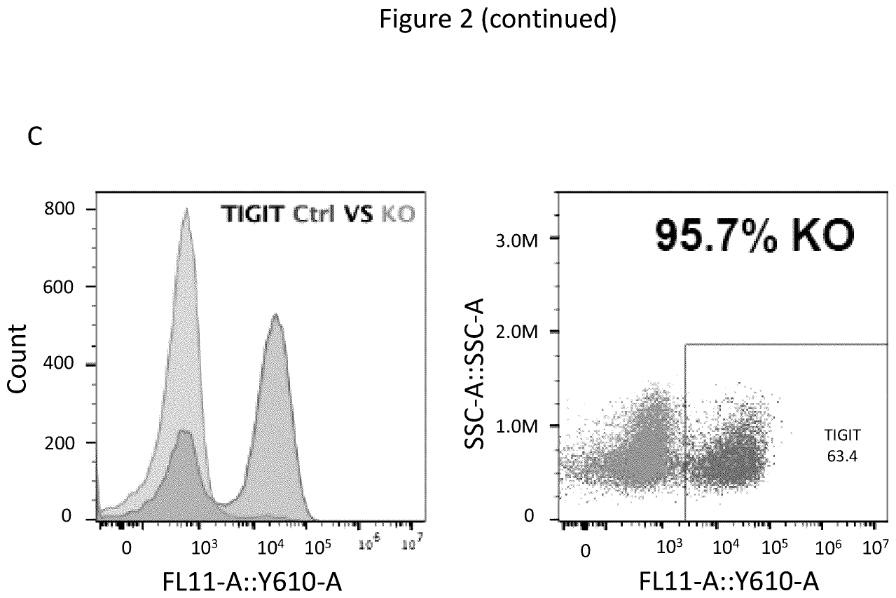

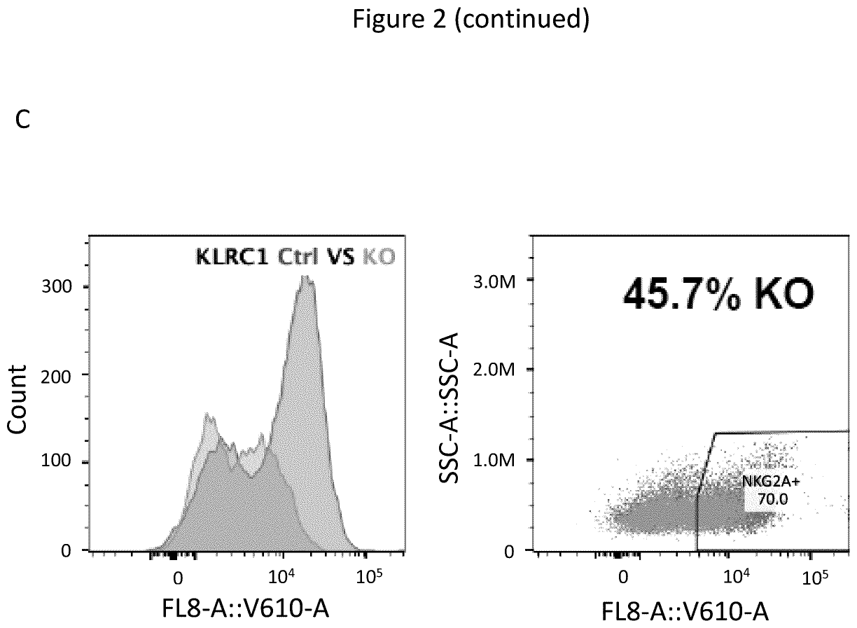

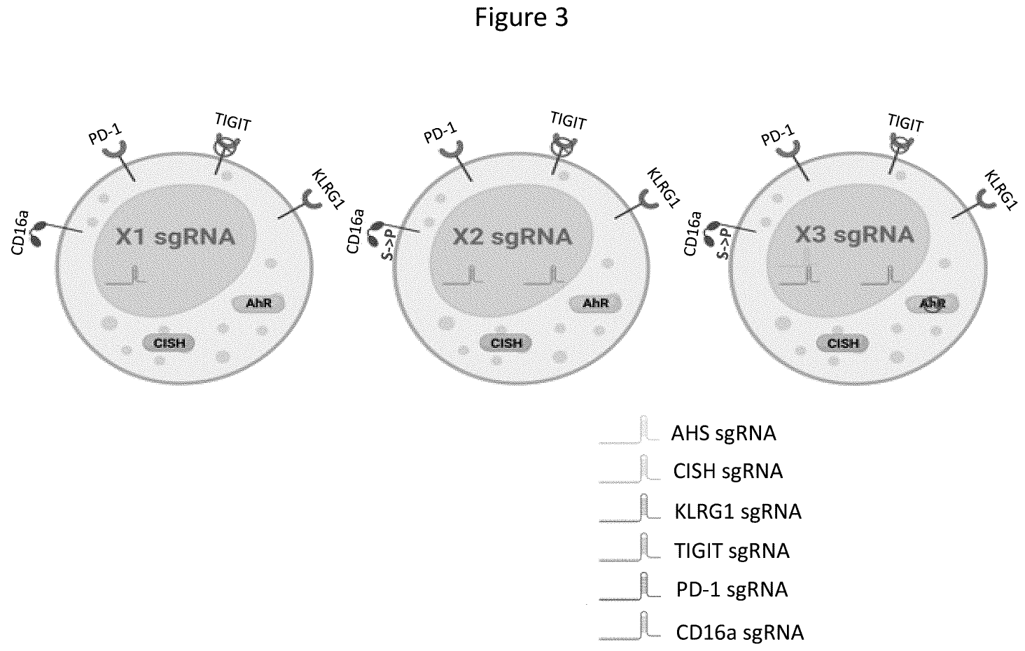

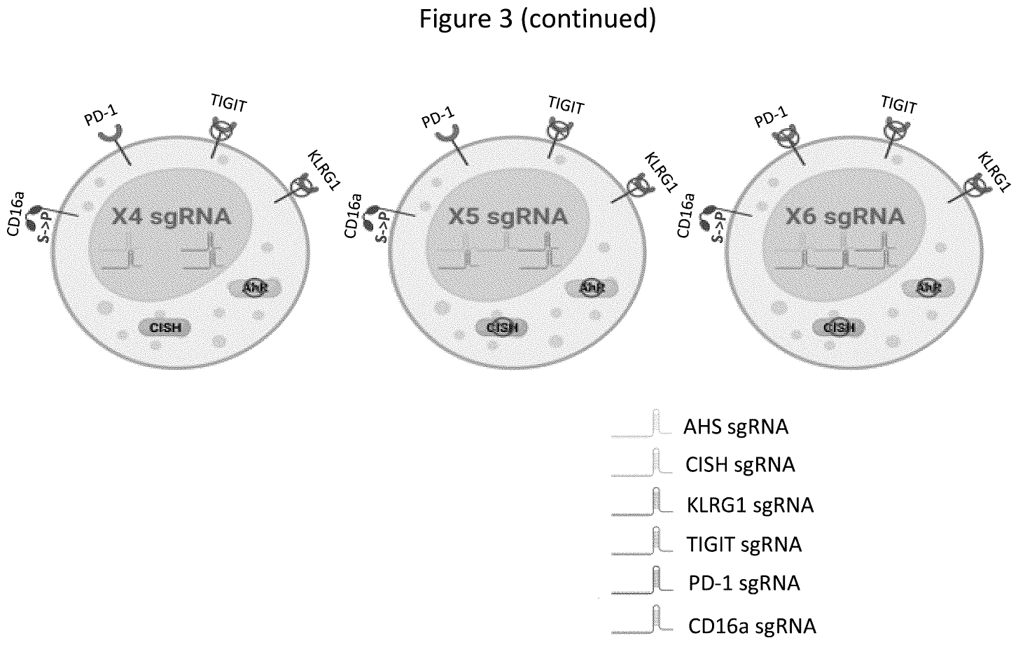

The gene editing methods of the present invention have been optimized to be highly efficient. As used herein, the term “efficiency” refers to the likelihood that an NK cell treated using the present methods will comprise the desired modification in the target sequence. For example, 80% efficiency means that when the method is applied to a population of NK cells, at least 80% of the treated NK cells comprise the desired modification. In Example 1, the inventors demonstrate that the methods can be used to achieve editing efficiencies of about 75%, 80%, or up to 100% at the DNA level (see FIG. 2A) and up to about of 80%, about 85%, and 99% at the protein level (see FIG. 2B) for the target genes AHR, CISH, KLRG1, TIGIT, KLRC1, and PD-1, while they achieved an editing efficiency of about 75% up to 98% at the DNA level (see FIG. 2A) and of at least 75%, and up to 98% or more at the protein level (see FIG. 2B) for the target gene CD16a. Thus, in some embodiments, the methods generate a targeted mutation in the NK cell with at least 60%, at least 65%, at least 70%, at least 75%, at least 80%, at least 85%, at least 90%, at least 95%, at least 96%, at least 97%, at least 98%, at least 99%, or 100% efficiency.

The methods may further comprise several additional steps. For example, in some embodiments, the methods further comprise selecting an NK cell that has been successfully modified. Suitable selection methods may include, for example, flow sorting (e.g., for GFP expression), magnetic bead separation (e.g., targeting a cell-surface marker), and transient drug resistance gene expression (e.g., antibiotic resistance). In some embodiments, the methods further comprise expanding the genetically modified NK cell by exposing it to a stimulating agent (e.g., an artificial antigen-presenting cell bound to IL-21).

The methods of the present invention may be used to prepare NK cells for downstream applications, including therapeutic applications. In some embodiments, the methods are performed in cell culture. For example, in some embodiments, the methods are performed ex vivo on NK cells that have been collected from a subject. In other embodiments, the methods are performed in vivo in a subject.

### Cells:

In a second aspect, the present invention provides genetically modified NK cells obtained according to the methods disclosed herein.

In some embodiments, the genetically modified NK cell includes a modification that alters the NK cell in one or more of the following ways relative to an unmodified NK cell: (1) alters cytokine or chemokine production (e.g., production of IFNγ, TNFα, IL-17, IL-22, MIP-1α (CCL3), MIP-1β (CCL4), and/or RANTES (CCLS)), (2) alters a cytokine receptor (e.g., IL-2R, IL-12R, IL-18R, IL-21R, etc.) or a chemokine receptor (e.g., CCR1, CCR2, CCR3, CCR4, CCR5, CCR6, CCR7, CXCR1, CXCR2, CXCR3, CXCR4, CXCR5, CXCR6, etc.), (3) alters a cytotoxic granule (e.g., Granzyme B and/or perforin) or a protein involved in the exocytosis of a cytotoxic granule (e.g., Wiskott-Aldrich Syndrome protein (WASp), WASp Interacting protein (WIP), Cdc42 Interacting protein-4 (CIP), Adaptor protein 3 complex (AP-3), Rab7 interacting lysosomal protein (RILP)/Rab7, Rab27a, Myosin IIa, Munc13-4, Syntaxin 11, VAMP7, Syntaxin 7, and/or Dynamin 2), (4) alters expression or activity of an activating receptor (e.g., CD16, CD94-NKG2C, NKG2D, 2B4, DNAM-1 (CD226), a member of the KIR2DS family, a member of the KIR3DS family, NKG2C, NKG2D, NKG2E, PILR (CD99), NKp30, NKp44, NKp46, NKp80, Sema4D (CD100), and/or CD160), (5) alters expression or activity of an inhibitory receptor (e.g., PD-1, CD94-NKG2A, NKG2A, TIGIT, CISH, a member of the KIR2DL family, a member of the KIR3DL family, KLRG1, LILR, 2B4 (CD48), CD96 (Tactile), LAIR1, KLB1 (CD161), CEACAM-1, SIGLEC3, SIGLEC7, SIGLEC9, and/or CTLA4), (6) alters expression or activity of an adaptor molecule (e.g., EAT2, DAP10, DAP12, and/or CD3zeta), (7) alters expression or activity of a downstream signaling molecule (e.g., ADAM17, a protein implicated in CD16 shedding, a tumor necrosis factor-related apoptosis-inducing ligand (TRAIL), and/or a cytotoxic effector molecule), or (8) introduces a non-endogenous gene (e.g., GFP).

In some embodiments, the genetically modified NK cell exhibits improved survival relative to an unmodified NK cell. For example, the modified NK cell may exhibit an increased capacity to proliferate (either in vivo, in vitro, or ex vivo) relative to an unmodified NK cell. Short in vivo persistence of NK cells has been one of the major drawbacks of NK-based cancer immunotherapies to date. As used herein, the term “persistence” is used to refer to the ability of an NK cell to remain active and/or proliferate in a tumor microenvironment. The methods of the present invention can be used to improve the in vivo persistence of an NK cell, for example, by modifying a gene to disrupt an immunosuppressive pathway, thereby making the NK cell more tolerant to a hostile tumor microenvironment. Thus, in some embodiments, the modified NK cells exhibit increased persistence in a tumor microenvironment relative to an unmodified NK cell.

In some embodiments, the genetically modified NK cell exhibits increased capacity to kill cancer cells relative to an unmodified NK cell. For example, the NK cell may exhibit increased antibody-dependent cellular cytotoxicity relative to an unmodified NK cell. Antibody-dependent cellular cytotoxicity (ADCC) is a mechanism of cell-mediated immune defense whereby an effector cell of the immune system actively lyses a target cell whose membrane-surface antigens have been bound by specific antibodies. In NK cells, ADCC is mediated by the surface receptor CD16a. During ADCC, the metalloprotease ADAM17 cleaves CD16a off the NK cells, leading to decreased NK cell functionality. A single amino acid substitution, S197P, has been shown to render CD16a non-cleavable by ADAM17, resulting in improved NK cell cytotoxicity during ADCC (PLOS One (2015) 10 (3):e0121788). Thus, in one embodiment, the genetically modified NK cell comprises a S197P substitution in CD16a and has improved ADCC relative to an unmodified NK cell.

In some embodiments, the genetically modified NK cell expresses a chimeric antigen receptor. As used herein, the term “chimeric antigen receptor (CAR)” refers to an artificially constructed fusion protein comprising an extracellular antigen binding domains of an antibody (e.g., single chain variable fragment (scFv)) operably linked to a transmembrane domain and at least one intracellular domain. Generally, the antigen-binding domain of a CAR has specificity for a particular antigen expressed on the surface of a target cell of interest. For example, NK cells can be engineered to express a CAR specific for CD19 on B-cell lymphoma.

In some embodiments, the genetically modified NK cell is a “universally acceptable” cell for a therapeutic application. As used herein, the term “universally acceptable” describes a cell produce that is generally acceptable in immunological terms, such that cross matching of patients and cells is not required, and no immunosuppression is needed. Such cells can be produced via disruption of immunogenic genes that make donor matching necessary.

In some embodiments the genetically modified NK cells described herein may be used for the treatment of viral infections, including but not limited to Hepatitis A, B, C, D, or E virus; Epstein Barr Virus; Cytomegalovirus; HIV; Human Papilloma Viruses, Human Herpes viruses; and other pathogens known to be targeted by NK cells.

The cells generated by the methods described herein can be used for several application, including studying NK cell biology and gene function, modeling diseases such as primary immunodeficiencies, correcting disease-causing point mutations, and generating novel NK cell products for therapeutic applications.

### Cancer Treatments:

Due to their innate ability to eliminate tumor cells, NK cell-based cancer immunotherapies have been investigated for decades. NK cells that have been genetically modified such that they have improved tolerance to immunosuppressive tumor microenvironments or have improved cytotoxicity (e.g., increased ADCC) may prove to be highly effective for such therapies. Thus, in a third aspect, the present invention provides uses of the genetically modified NK cells disclosed herein in a treatment for cancer.

As used herein, the term “cancer” refers to an abnormal mass of tissue in which the growth of the mass surpasses and is not coordinated with the growth of normal tissue. In the case of hematological cancers, this includes a volume of blood or other bodily fluid containing cancerous cells. The methods of the present invention can be used to treat any type of cancer including, but not limited to, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CIVIL), Hodgkin's disease, and/or multiple myeloma. The cancer may be a metastatic cancer. The cancer may comprise a solid tumor and/or hematological malignancies.

As used herein, “treating” or “treatment” describes the management and care of a subject for the purpose of combating a disease, condition, or disorder. Treating includes administering a treatment to prevent the onset of the symptoms or complications, to alleviate the symptoms or complications, or to eliminate the disease, condition, or disorder. For example, treating cancer in a subject includes the reducing, repressing, delaying or preventing of cancer growth, reduction of tumor volume, and/or preventing, repressing, delaying or reducing metastasis of the tumor. Treating cancer in a subject also includes the reduction of the number of tumor cells within the subject.

The genetically modified NK cell may be administered to a subject alone or in combination with one or more other therapies, such as pharmaceutical compositions (e.g., a chemotherapeutic agent, an anti-tumor antibody, an NK cell receptor ligand), cellular therapies (e.g., chimeric antigen receptor T cells, stem cells), hormone therapies, radiation, or surgery. The genetically modified NK cell may be administered to the subject before, during, and/or after another therapy. Administration of the NK cell may be separated in time from the administration of other therapies by hours, days, or even weeks.

The NK cell may be administered in a variety of routes, including, for example, intravenously, intratumorally, intraarterially, transdermally, via local delivery by catheter or stent, via a needle or other device for intratumoral injection, subcutaneously, etc. The NK cell may be administered once or multiple times. A physician having ordinary skill in the art may determine and prescribe the effective amount and dosing of an NK cell.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

### Additional Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1. High Efficiency Method for Gene Editing in Natural Killer (NK) Cells

In the following example, the inventors describe their method for genetically modifying natural killer (NK) cells and demonstrate that this method produces modified NK cells with high efficiencies.

### Genes of Interest (GOIs):

AHR: aryl hydrocarbon receptor
   Rationale: AHR is an inhibitory intracellular regulator. AHR expression gradually decreases as NK cells mature. Knocking out AHR causes NK cell to display a more mature phenotype.
CISH: cytokine-inducible SH2-containing protein
   Rationale: CISH is an inhibitory intracellular regulator. CISH inhibits IL-15 signaling, which stimulates multiple NK cell functions. Knocking out CISH improves NK cell function and makes NK cells less sensitive to “IL-15 limiting” conditions.
KLRG1: killer cell lectin-like receptor subfamily G member 1
   Rationale: KLRG1 is an inhibitory checkpoint receptor. The expression level of KLRG1 is inversely correlated with an NK cell's ability to proliferate and levels of IFN-γ production. Knocking out KLRG1 increases the IFN-γ production and proliferative capacity of NK cells.
TIGIT: T cell immunoreceptor with Ig and ITIM domains
   Rationale: TIGIT is an inhibitory checkpoint receptor. Knocking out TIGIT in NK cells prevents NK cells exhaustion.
KLRC1: killer cell lectin-like receptor subfamily C member 1, aka NKG2A
   Rationale: KLRC1 is an inhibitory checkpoint receptor. Knocking out KLRC1 overcomes tumor resistance in NK cells.
PDCD1: programmed cell death protein 1
   Rationale: PDCD1 is a classic inhibitory checkpoint receptor.
CD16A: Fc receptors FcγRIIIa
   Rationale: In NK cells, antibody-dependent cellular cytotoxicity (ADCC) is mediated by the surface receptor CD16a. During ADCC, the metalloprotease ADAM17 cleaves CD16a off the NK cells (a process referred to as CD16a shedding), leading to decreased NK cell functionality. A single amino acid substitution, S197P, has been shown to render CD16a non-cleavable by ADAM17, resulting in improved NK cell cytotoxicity during ADCC (PLOS One (2015) 10 (3):e0121788).
      Single Guide RNA (sgRNA) Design:

We designed multiple sgRNAs for each GOI and tested them in K562 cells. Top candidates for each GOI were tested in for single or multiplex editing in NK cells. These sequences are provided in Table 1 below.

TABLE 1
sgRNA sequences

| Name | Sequence |
|---|---|
| AHR Ex.2 SD ABE sgRNA | CTTACCATCAAAGAAGCTCT (SEQ ID NO: 1) |
| CISH Ex.2 SD ABE sgRNA | CTCACCAGATTCCCGAAGGT (SEQ ID NO: 2) |
| KLRG1 Ex.4 SD ABE | CCTTACCTTGAGAAGTTTAG (SEQ ID NO: 3) |
| sgRNA | |
| TIGIT Ex. 1 SD ABE sgRNA | CAGGCCTTACCTGAGGCGAG (SEQ ID NO: 4) |
| KLRC1 (NKG2A) Ex.3 SD | TGCTTTACCTTTGCAGTGAT (SEQ ID NO: 5) |
| ABE sgRNA | |
| PD1 Ex.1 SD ABE sgRNA | CACCTACCTAAGAACCATCC (SEQ ID NO: 6) |
| CD16A (S197P) sgRNA | TTGACACTGCCAAACCTATT (SEQ ID NO: 7) |

*Ex.2: Exon 2; SD: splice donor; ABE: adenine base editor
*Note:
The sgRNAs provided in this table are designed for use with the base editor ABE8e, which edits a target sequence on the strand opposite to the strand that the sgRNA binds to. Thus, the sgRNA sequences provided in the table are identical to the protospacer sequence to be edited.

### Gene Editing Protocol:

### Materials:

1. Stimulated NK cells: 3e5/sample for 10 uL zap, 3e6/sample for 100 uL zap
2. RNA: ABE8 mRNA, sgRNAs
3. Protector RNase Inhibitor (Millipore Sigma Cat #3335402001)
4. Alt-R® Cas9 Electroporation Enhancer (IDT)
5. 24-well plate (for 10 uL zap) and 6-well plate (100 uL zap)
6. Neon™ Transfection System Kit (10 uL and 100 uL)
7. NK recovery media (AIM-V+5% ICSR+100 IU/mL IL-2, no Pen/Strep)

### Protocol:

1. Prepare plates with medium for pre-warm
   a. 24-well plate w/580 uL NK recovery media (if using 10 uL tips, but twice) 6-well plate with 2.9 mL NK recovery media (if using 100 uL tips)
   b. Plate layout:
      Depends on the actual experimental design, but include an ABE8e mRNA only control
2. Count cells
3. Transfer all the NK cells needed to a 15 ml conical tube
4. Spin 400×g for 5 minutes and pour off supernatant
5. Wash cells with PBS without Ca2+ or Mg2+, spin at 400×g for 5 minutes and pour off supernatant
6. Resuspend cells at 7 uL/300K (for 10 uL zap) and 70 uL/3e6 (for 100 uL zap) in Buffer T (for primary T and B cells, PBMCs, monocytes, and bone marrow-derived cells) and mix by pipetting
7. Aliquot cells into sterile microtubes for each transfection
   a. 7 uL per sample for 10 uL zap
   b. 70 uL per sample for 100 uL zap
8. Add appropriate nucleic acid to tubes (For the ABE8e mRNA only control: add everything listed below except for the sgRNAs)
   a. ABE8 (3 ug/uL): 0.5 uL per sample for 10 uL tips; 5 uL for 100 uL tips
   b. sgRNA(s) (2 nM): 0.5 uL/sample for 10 uL tips; 5 uL/sample for 100 uL tips (high conc sgRNA)
   c. RNase inhibitor: 1 uL of 1:5 diluted solution (diluted in T buffer) from stock for 10 uL tips; 2 uL of stock for 100 uL tips (#incubate at RT for 5 mins)
   d. Electroporation enhancer: 1 uL of 1:5 diluted solution (diluted in T buffer) from stock for 10 uL tips; 2 uL of stock for 100 uL tips
   e. Final volume for 10 uL should be 12 uL and for 100 uL should be 120 uL (to avoid bubbles). Use T buffer to fill up to the volumes.
9. Place a Neon cuvette into the machine and fill with 3 mL buffer E (10 uL tip) or buffer E2 (100 uL tip)
10. Grab a Neon tip with the Neon pipette and draw up cell/nucleic acid mixture (make sure to mix properly, and check for air bubbles in the tip—this will cause arcing and kill cells)
11. Proceed with transfection using Neon machine, use the saved program for NK ABE8e electroporation: pulse voltage 1825V, pulse number 2, pulse width 10 ms.
12. Place transfected cells into the wells of pre-warmed medium and incubate at 37° C. for 72 hrs
13. After 72 hrs, harvest cells for analysis or do an NK expansion to expand cell numbers

### Assessment of Editing Efficiency:

At the DNA level:
   Phire Tissue Direct PCR Master Mix (Thermo Scientific Cat #F170S) is used to extract DNA and amplify our gene of interest using PCR.
   PCR product is purified and sent for Sanger sequencing.
   Base editing efficiencies are determined by analyzing the sequencing results using EditR (baseeditr.com; CRISPR J (2018) 1 (3): 239-250).
At the protein level:
   The knockout (KO) efficiency of KLRG1, TIGIT, KLRC1 and PDCD1, which are surface proteins, are evaluated using flow cytometry.
   The KO efficiency of AHR and CISH, which are intracellular proteins, are evaluated using western blotting.
   The efficiency of non-cleavable CD16A, which is a surface protein, is evaluated by propidium monoazide (PMA) treatment followed by flow cytometry.
      Rationale: PMA activation of NK cells induces ADAM17 activity, which leads to increased CD16a shedding. When CD16a is edited to the non-cleavable form (S197P), ADAM17 cannot remove CD16a from the NK surface. Thus, PMA treatment allows us to confirm that CD16a has been edited.
Functional data:
   For AHR, CISH, TIGIT, KLRG1, and PDCD1 KO, functional evaluation is confirmed by co-culturing edited NK cells with relevant cancer cell lines.
   For CD16A editing, we used an antibody-dependent cellular cytotoxicity (ADCC) assay to test the cleavage resistance of the CD16a (S197P) mutant.

### Results:

First, we tested whether the inclusion of an RNase inhibitor in our transfection mix would affect the editing efficiency achieved with our method. As is shown in FIG. 1, inclusion of the RNase inhibitor significantly increased the editing efficiency.

Next, we tested the ability of our method to produce NK cells with single gene knockouts. mRNA encoding the base editor ABE8e and one of the sgRNAs provided in Table 1 were introduced into primary human NK cells. As is shown in FIG. 2, the editing efficiency at the individual target genes AHR, CISH, KLRG1, TIGIT, KLRC1, and PD-1 was found to be up to 100% at the DNA level (FIG. 2A), and was found to be up to 99% at the protein level (FIG. 2B). As is shown in FIG. 2, the editing efficiency at the target gene CD16a was found to be up to 98% at the DNA level (FIG. 2A), and was found to be up to 98% at the protein level (FIG. 2C).

We then tested the ability of our method to be used for multiplex editing. mRNA encoding ABE8e and the sgRNAs for six target genes (i.e., AHR, CISH, KLRG1, TIGIT, PD-1, and CD16a) were simultaneously introduced into primary human NK cells. As is shown in FIG. 4, the multiplex editing efficiency was found to be up to 100% at the DNA level (FIG. 4A) and was found to be 99% at the protein level (FIG. 4B). Importantly, this efficiency was not significantly affected by the number of genes being knocked out

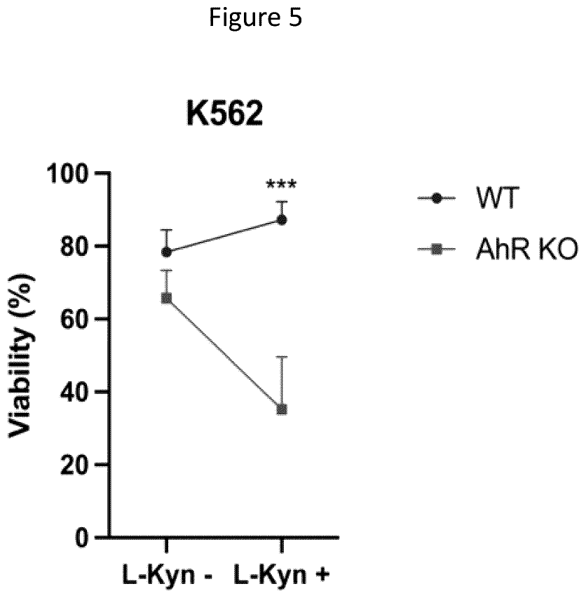

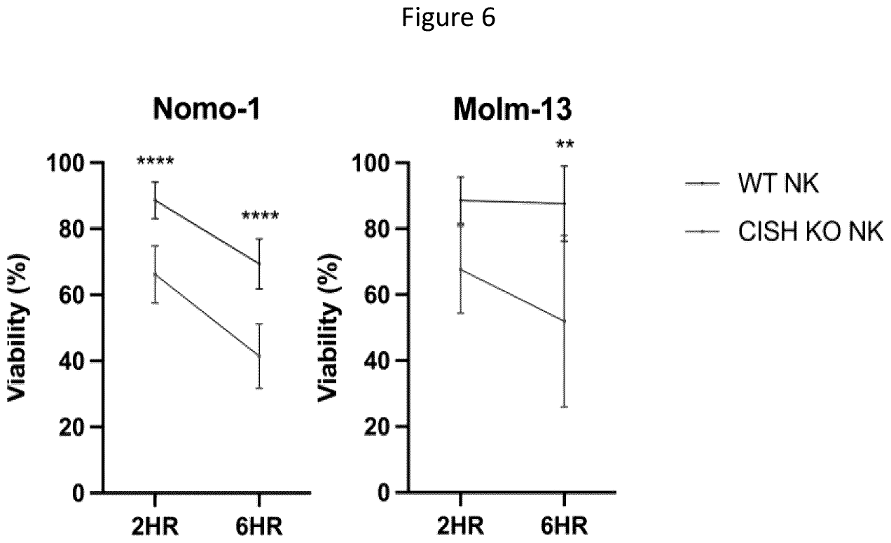

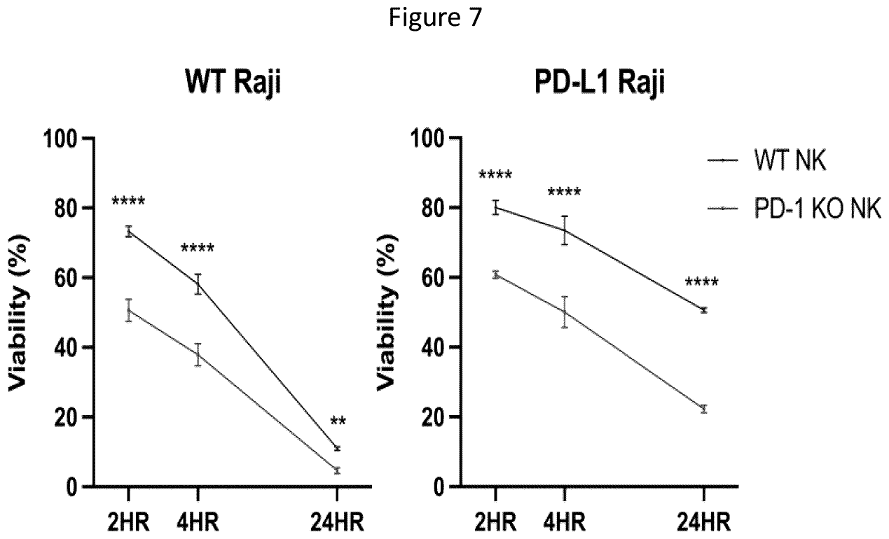

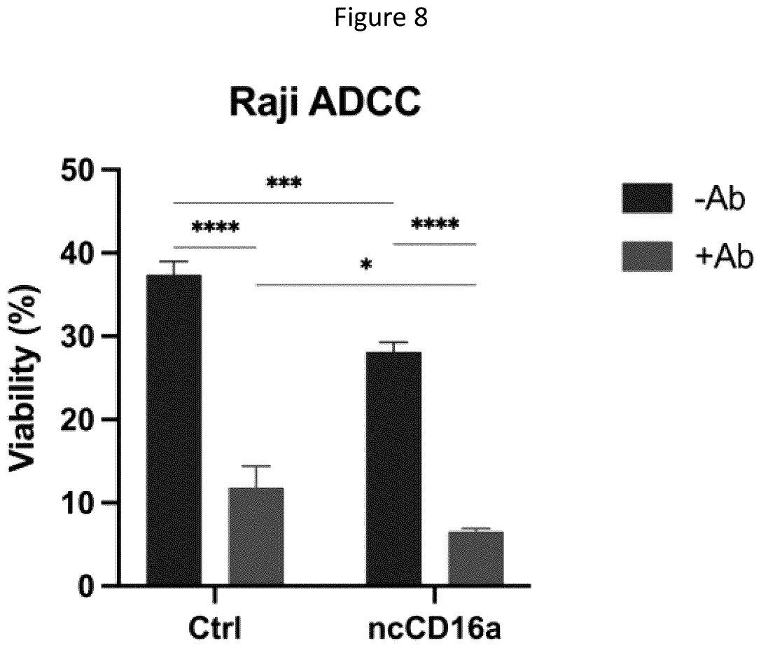

Finally, we tested the editing of each target gene at functional level. Genetically modified NK cells were co-cultured with relevant cancer cells and their cytotoxicity was evaluated. As is shown in FIG. 5-8, genetically modified NK cells demonstrated improved cytotoxicity against cancer cells when compared to the unmodified NK cells.

### Example 2. Generation of Dominant Negative Fas and TGFbR2 Natural Killer (NK) Cells

FasL and transforming growth factor β (TGF-β) are commonly upregulated in cancer cells. FasL is a ligand that directly kills the natural killer (NK) cells by inducing apoptosis, while TGF-β is immunosuppressant cytokine that is secreted by cancer cells. Thus, cancer cells that express these proteins generate a hostile microenvironment that shields the tumor from immunosurveillance.

However, T cells with dominant negative mutations in Fas (i.e., the receptor for FasL) have been shown to have improved anti-cancer capabilities and better survival than wild-type T cells due to their resistance to FasL signaling (J Clin Invest (2019) 129 (4): 1551-1565). Likewise, CAR T-cells and NK cells with dominant negative mutations in TGF-β receptor II (referred to herein as TGFbR2) have been shown to have improved cancer-clearing capabilities in the presence of TGF-β-secreting tumors as compared to wild-type cells (Mol Ther (2018) 26 (7): 1855-1866; Cytotherapy (2017) 19 (3): 408-418).

Thus, in this example, the inventors use their method for high efficiency editing of NK cells to generate genetically modified NK cells that have dominant negative mutations in the genes Fas and TGFbR2. These mutations are expected to improve the performance of NK cells in the tumor microenvironment, allowing them to kill cancer cells in the hostile immunosuppressive microenvironment created by FasL- and TGF-expressing cancer cells. Thus, these modified NK cells represent a promising immunotherapy for the treatment of such cancers.

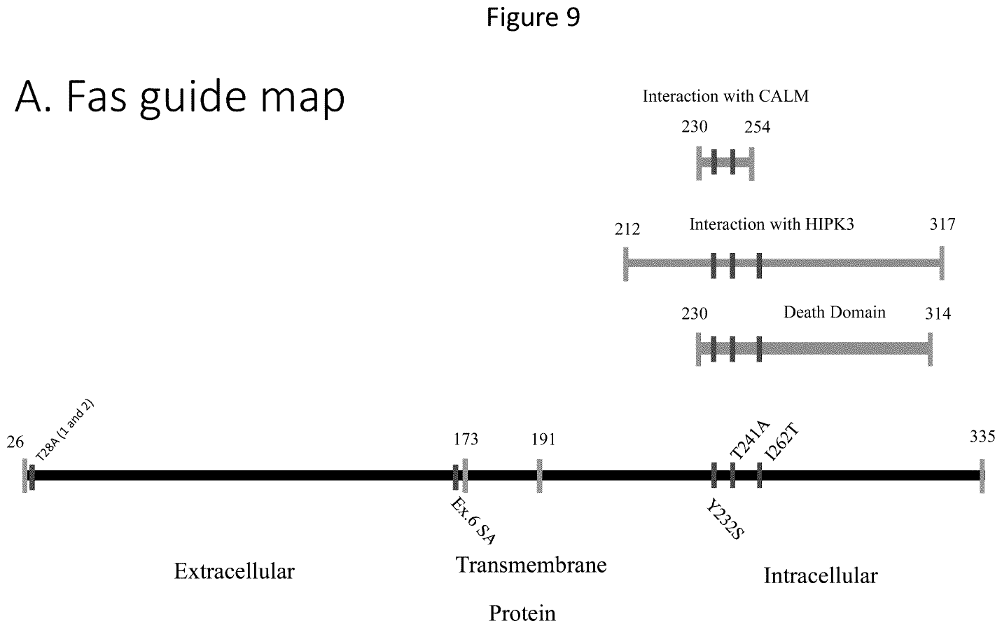

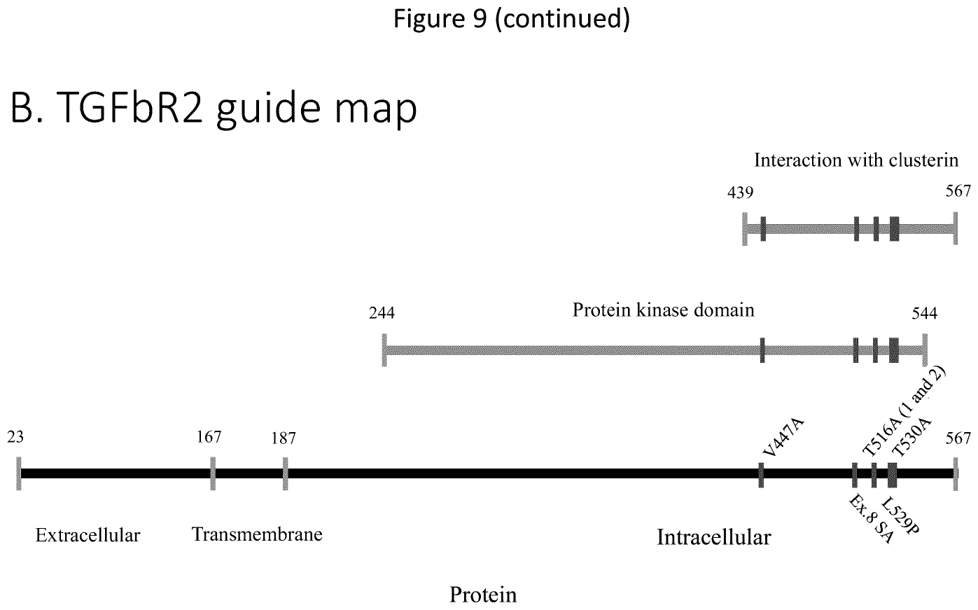

Specifically, the inventors have designed sgRNAs to make mutations in Fas that disrupt splicing (i.e., in exon 6) or create mutations that mimic those seen in autoimmune lymphoproliferative syndrome (ALPS; i.e., T28A, Y232S, T241A, I262T). For TGFbR2, they have designed sgRNAs to make mutations that disrupt splicing (i.e., in exon 8) or create mutations that mimic those seen in Loeys-Dietz syndrome type II (LDS2; i.e., T516A, L529P, T530A, V447). Maps showing the locations of these mutations within Fas and TGFbR2 are provided in FIG. 9. The sequences of the sgRNAs designed generate these mutations are provided in Table 2.

TABLE 2
sgRNA sequences

| Name | Sequence | Nuclease | Notes |
|---|---|---|---|
| FAS Ex.9 | AAATATATCACCACTATTGC | ABE | Y232C is observed in ALPS1A, |
| Y232S | (SEQ ID NO: 8) | | although it does not affect |
| | | | interaction with FADD. |
| FAS Ex.9 | ATGACACTAAGTCAAGTTAA | ABE/CBE | Two mutations at position 241 |
| T241A | (SEQ ID NO: 9) | | are associated with ALPS1A. |
| | | | CBE will produce T241I. |
| FAS Ex.9 | ATTCTTGATCTCATCTATTT | ABE | I262S is seen in ALPS1A. |
| I262T | (SEQ ID NO: 10) | | |
| FAS Ex.6 | TACAGGATCCAGATCTAACT | ABE | Disrupting splicing here may |
| SA | (SEQ ID NO: 11) | | cause issues since it is close to |
| | | | the transmembrane region. |
| FAS Ex.2 | AGTGACTGACATCAACTCCA | ABE/CBE | This exact mutation is seen in |
| T28A 1 | (SEQ ID NO: 12) | | ALPS1A. CBE will produce |
| | | | T28I. |
| FAS Ex.2 | GTGACTGACATCAACTCCAA | ABE/CBE | This exact mutation is seen in |
| T28A 2 | (SEQ ID NO: 13) | | ALPS1A. CBE will produce |
| | | | T28I. |
| TGFBR2 | GTGAGACGTTGACTGAGTGC | ABE/CBE | CBE will produce T516M. |
| Ex.8 | (SEQ ID NO: 14) | | |
| T516A 1 | | | |
| TGFBR2 | TGAGACGTTGACTGAGTGCT | ABE/CBE | CBE will produce T516M. |
| Ex.8 | (SEQ ID NO: 15) | | |
| T516A 2 | | | |
| TGFBR2 | GGGCTGTGAGACGGGCCTCT | ABE | Position 529 is beside two |
| Ex.8 | (SEQ ID NO: 16) | | positions known to be mutated in |
| L529P | | | LDS2. |
| TGFBR2 | CCGTCTCACAGCCCAGTGTG | ABE/CBE | T530I is observed in LDS2. CBE |
| Ex.8 | (SEQ ID NO: 17) | | will produce T530I. |
| T530A | | | |
| TGFBR2 | CCCGCTACAGGGCATCCAG | ABE | Exon 8 is very important for |
| Ex.8 SA | A (SEQ ID NO: 18) | | TGFBR2 function |
| TGFBR2 | GTAGACATCGGTCTGCTTGA | ABE/CBE | This exact mutation is observed |
| Ex.6 | (SEQ ID NO: 19) | | in breast cancer and inhibits |
| V447A | | | TGFb signaling. CBE will |
| | | | produce V447I. |

*Note:
The sgRNAs provided in this table are designed for use with the base editor ABE8e, which edits a target sequence on the strand opposite to the strand that the sgRNA binds to. Thus, the sgRNA sequences provided in the table are identical to the protospacer sequence to be edited.

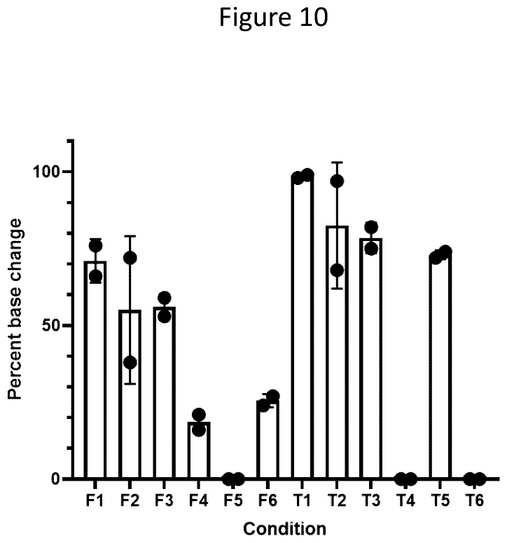

FIG. 10 shows base editing guides for FAS (F) and TGFβR2 (T). F1, 2, & 3 showed the greatest percent base editing for Fas, while T1, T2, T3 and T5 showed the most for TGFβR2, with all 7 sgRNA having at least 50% base conversion.

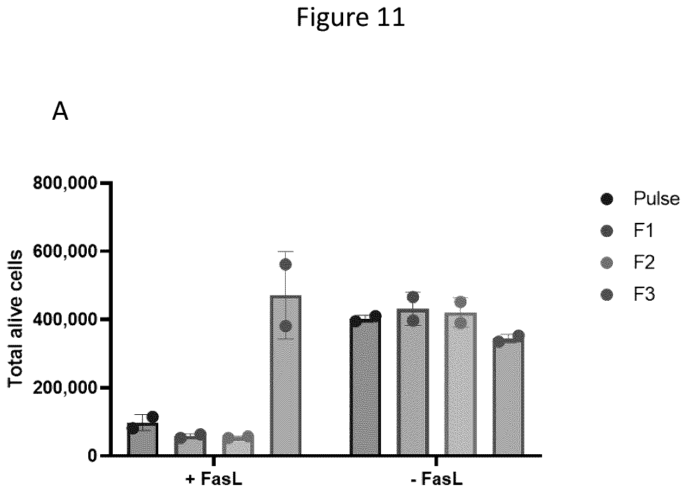

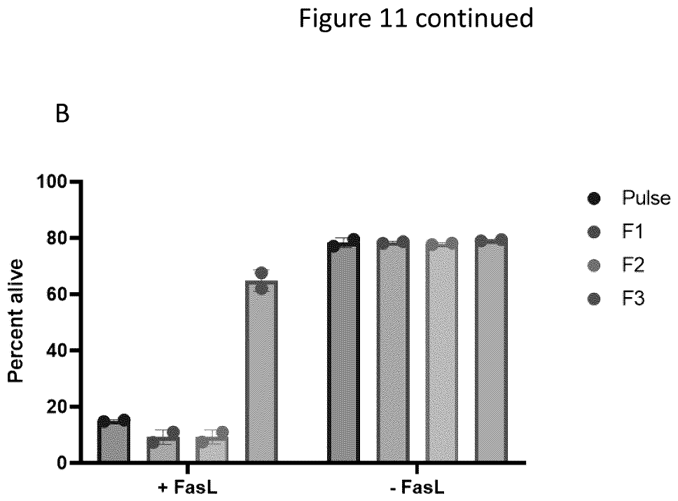

Guides with greater than 50% base conversion were used in FIG. 11. F3 was the most successful at preventing the effects of Fas ligand and preventing cell death.

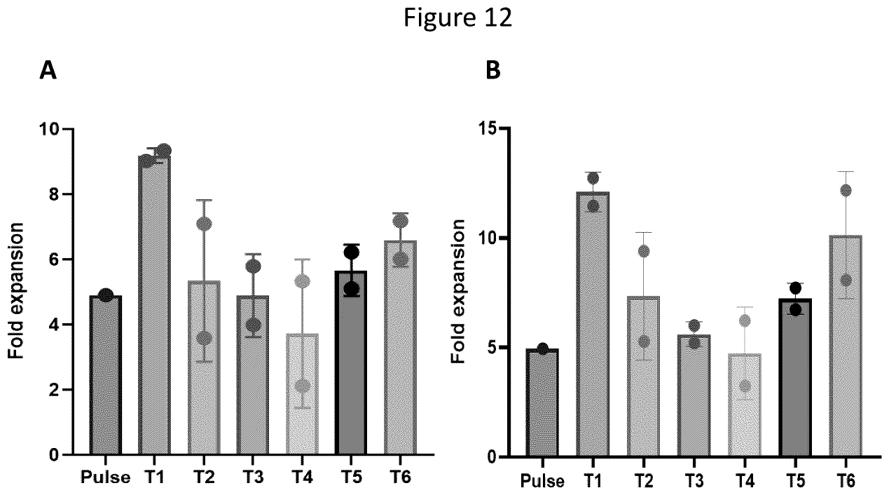

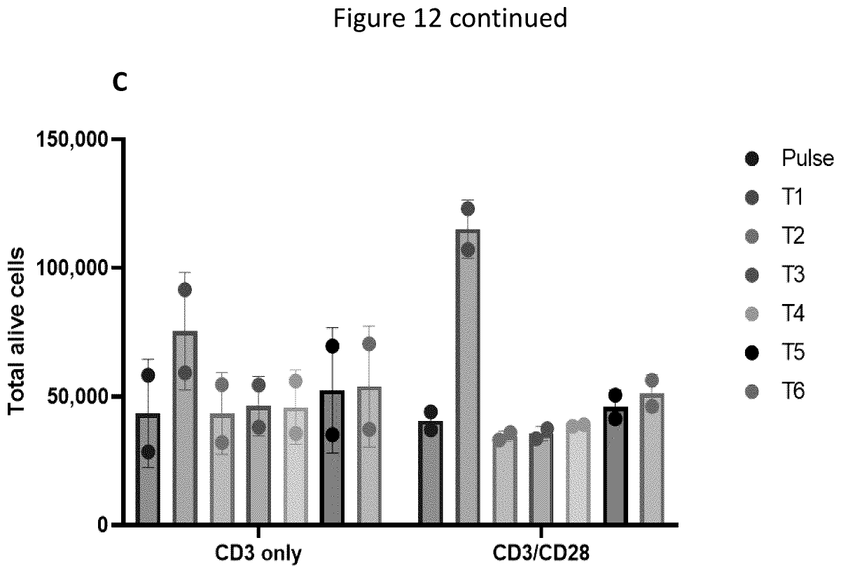

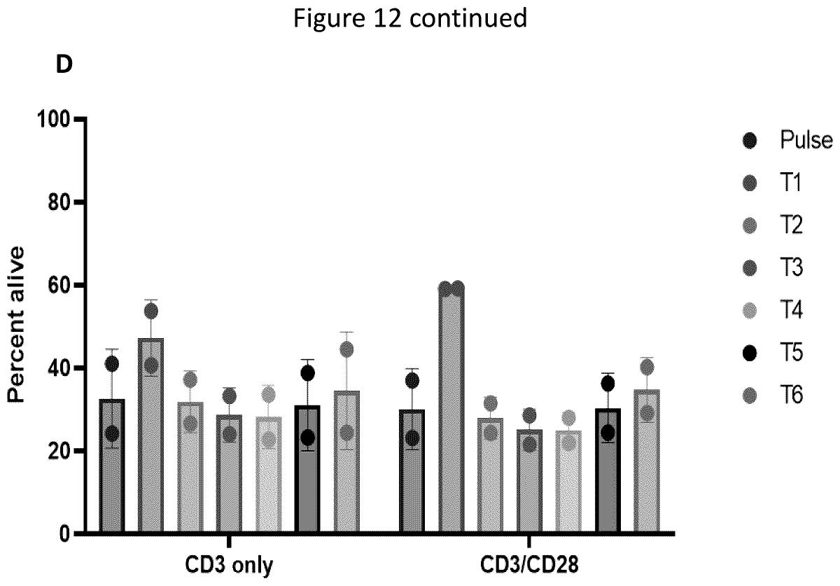

Cell growth in the presence of TGFβ was measured in FIG. 12. Cells edited with the T1 guide exhibit enhanced proliferation compared to cells treated with the other guides and pulse control at both day 8 and 13 post 1^{st} restimulation and day 4 post 3^{rd} restimulation. Immunosuppressive effects of TGFβ cause cell growth to slow, however the growth of T cells with T1 demonstrate T1 is inducing resistance to the immunosuppressive effects of TGFβ. These results confirm that T1 is working to make TGFβR2 dominant negative.

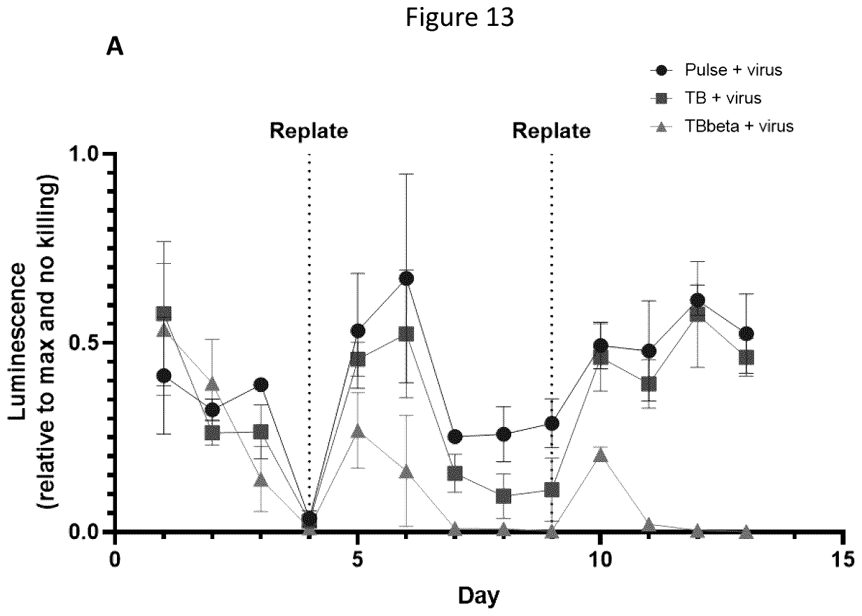

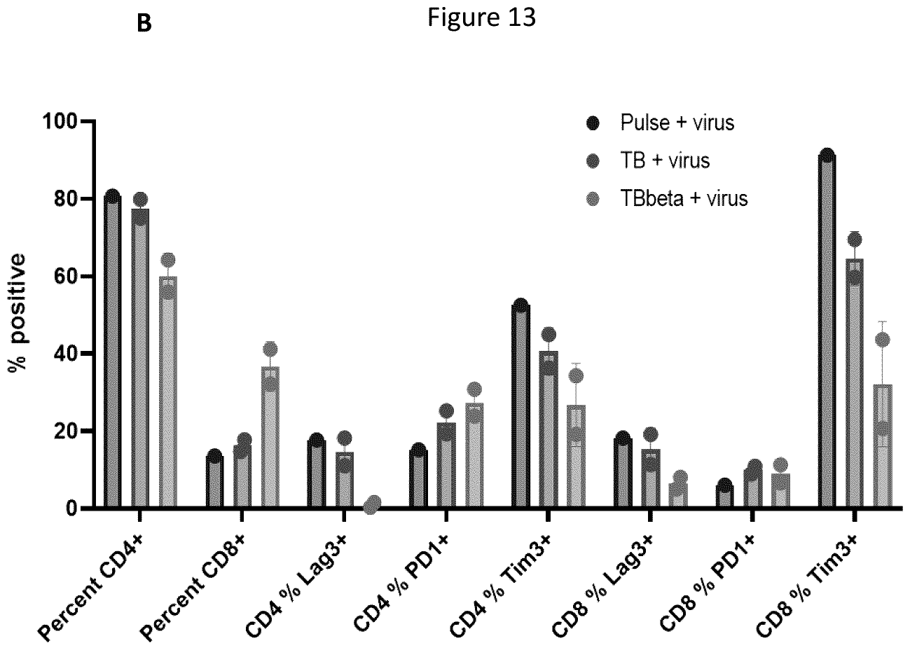

Last we measured the ability of DN TGFβR2 CAR T cell to kill Raji target cells. As shown in FIG. 13, initially all three conditions killed similarly. However, after the first replating, pulse and TB cells stopped killing, while TBbeta cells continued to kill. Cells were then replated again and TBbeta cells continued to kill target cells, while the other two conditions were unable. FIG. 13A further shows TBbeta cells were able to rebound and kill faster after each replate. An exhaustion panel was performed five days after the first replate, and as shown in FIG. 13B TBbeta cells exhibited a less exhaustive phenotype, as shown by decreased Lag3 and Tim3 on CD4 and CD8 cells, increased PD1 on CD4 cells and an overall increase in the percent of CD8 cells. Together this exciting data show that DN TGFβR2 prevents the T cells from becoming exhausted in the presence of TGFβ, allowing them to kill more target cells than WT TGFβR2 T cells.

### Methods

### T Cell Culture Media

The media used for T cell culture is a blend of the following:
91.8% (400 mL) OpTmizer T-Cell basal expansion medium (Gibco Cat #A10221-01)
2.38% (10.4 mL) OpTmizer T-Cell expansion supplement (Gibco Cat #A10484-02)
2.3% (10 mL) CTS Immune Cell serum replacement (Gibco Cat #A25961-01)
0.91% L-Glutamine (Gibco Cat #25030-081)
0.91% Penicillin/Streptomycin (Millipore Cat #TMS-AB2-C)
7.3 mL (10 mM) N-Acetyl-cysteine (Sigma Cat #A9165-256)
300 IU/mL Recombinant Human IL-2 (Peprotech Cat #200-02)
5 ng/ml Recombinant Human IL-7 (Peprotech Cat #200-07)
5 ng/ml Recombinant Human IL-15 (Peprotech Cat #200-15)
   Recovery media is the same as above but without cytokines (IL-2, IL-7, and IL-15) or antibiotics (Pen/Strep)

### T Cell Engineering

Primary human T cells from two independent donors were stimulated with anti-CD3/CD28 Dynabeads for 48 hours before electroporation at a density of 1e6 cells per mL and a 2:1 bead to cell ratio. If applicable, 24 hours before electroporation, the T cells were transduced with Lentivirus for CAR knock-in. This was done at an MOI of 20 with 0.25 mg/mL F108 added beforehand. Before electroporation, the Dynabeads were removed magnetically. The electroporation machine used was the Lonza Nucleofector 4D and the kit used was the P3 primary cell kit. Per electroporation condition, 1e6 T cells were resuspended in 80 ul of the transfection solution from the kit. 1 ug of each guide RNA and 1.5 ug of ABE8e mRNA were added. The electroporation code was EO-115. After electroporation, the cells were left alone for 15 minutes at room temperature before being moved to recovery media for 15 minutes at 37 C. From here, regular T cell media was added as well as more Dynabeads, this time at a 1:2 bead to cell ratio. The next day, the T cells were moved to a G-Rex culture system and were allowed to grow for 7 days. At this point, the Dynabeads were removed magnetically and the cells were frozen down for later downstream analysis.

### Assessment of Editing Efficiency

1e6 T cell pellets were taken from edited T cells and the gDNA was extracted using the Thermo Scientific GeneJET Genomic DNA Purification Kit (Thermo Cat #K0722) according to the manufacturer's protocol. After this, the site of interest was amplified using PCR. The site of interest is where base editing would occur and is based off of where the particular gRNA is directing ABE8e to the genome. The PCR products were purified using the Qiagen QIAquick PCR purification kit (Qiagen Cat #28106) according to the manufacturer's protocol. The purified PCR products were sequenced by Sanger sequencing and the results were analyzed using EditR (baseeditr.com; CRISPR J (2018) 1 (3): 239-250).

### Fas Ligand Killing Assay

1e6 T cells were plated in wells of a 24-well plate and exposed to 10 ug/mL of His Tag antibody (R&D Systems Cat #MAB050) and 300 ng/ml Fas ligand (R&D Systems Cat #126-FL-010) for 24 hours. Cell viability was determined using Trypan blue stain.

### TGFβ Growth Assay

1e6 T cells were stimulated with plate-bound CD3 antibody (Invitrogen Cat #16-0037-85) or plate-bound CD3 antibody and soluble CD28 antibody (Invitrogen Cat #16-0289-85) in a 24-well plate for two days. They were then moved to a clean plate with no antibodies. After another two days, they were moved to a 24-well G-Rex culture system and TGFβ (Bio-Techne Cat #7754-BH-025) was added to the media at 25 ng/mL. After eleven days under TGFβ, the cells were counted and 2 mL of cells were moved to a fresh 24-well plate with TGFβ and plate-bound CD3 alone or TGFβ, plate-bound CD3, and soluble CD28 antibody for the first restimulation. The cells were counted using the Invitrogen Countess II machine. After two days here, the cells were moved to another 24-well G-Rex. The cells were allowed to sit for nineteen days, with TGFβ being replenished every 2 days. At day zero, eight, and sixteen from this first restim, 50 k cells were moved from the G-Rex to a 96-well plate coated with plate-bound CD3 antibody, TGFβ in the media, and with or without CD28 antibody. These were allowed to sit for four days before flow cytometry analysis, with media and TGFβ being replenished on day two.

### TGFβ CAR-T Luciferase Serial Killing Assay

T cells engineered with a CD19-specific CAR were co-cultured with Raji target cells expressing Luciferase at a 3:1 effector to target ratio. There were 100 k Raji-Luc cells in each well of a 96-well opaque plate, and TGFβ (Bio-Techne Cat #7754-BH-025) was added to the media at 25 ng/mL. The media used for this assay was RPMI 1640 supplemented with 10% FBS, 100 Units/mL Penicillin, and 100 ug/mL Streptomycin. Every 24 hours, 280 ng/mL D-Luciferin was added to the media and luminescence was read on a plate reader after 10 minutes. The TGFβ was replenished every two days, along with a half media change. Once the T cells killed all of the targets, they were collected, counted, and replated in a new plate with new target cells for another round of killing. This went on until the T cells stopped killing. No killing was determined from wells with only targets and no T cells, and max killing was determined from wells with only targets and no T cells and 0.1% Triton-X added to the media.

What is claimed:

1. A method for producing a genetically modified natural killer (NK) cell, the method comprising introducing a combination of editing reagents into a NK cell, the editing reagents comprising:
a) a base editor protein or a plasmid or mRNA encoding the base editor; and
b) one or more guide RNAs (gRNAs) that comprise a sequence that is complementary to a target sequence within the genome of the NK cell;
thereby generating a genetically modified NK cell in which the target sequence is modified, wherein an RNase inhibitor is added to the editing reagents before they are introduced into the NK cell.

2. (canceled)

3. The method of claim 1, wherein introduction of editing reagents is accomplished by electroporation.

4. The method of claim 3, wherein an electroporation enhancer is added to the editing reagents prior to electroporation.

5. The method of claim 4, wherein the electroporation enhancer is a carrier DNA.

6. The method of claim 3, wherein the electroporation comprises exposing the NK cell to 1700-2000 volts and at least 2 energy pulses with a pulse width of 10 milliseconds.

7. The method of claim 1, wherein at least two gRNAs are introduced into the NK cell, and wherein each of the gRNAs is designed to target a different sequence within the genome of the NK cell, such that the method generates a genetically modified NK cell in which at least two target sequences have been modified.

8. The method of claim 1, wherein the target sequence is within a target gene selected from: aryl hydrocarbon receptor (AHR), cytokine-inducible SH2-containing protein (CISH), killer cell lectin-like receptor subfamily G member 1 (KLRG1), T cell immunoreceptor with Ig and ITIM domains (TIGIT), killer cell lectin-like receptor subfamily C member 1 (KLRC1), programmed cell death protein 1 (PDCD1), CD16A, Fas, and transforming growth factor beta receptor 2 (TGFBR2).

9. (canceled)

10. The method of claim 8, wherein the target gene is Fas and the genetically modified NK cell comprises a mutation in Fas selected from T28A, Y232S, T241A, and I262T.

11. The method of claim 8, wherein the target gene is TGFBR2 and the genetically modified NK cell comprises a mutation in TGFBR2 selected from T516A, L529P, T530A, and V447.

12. The method of claim 1, wherein the method generates a dominant negative mutation in the NK cell.

13. The method of claim 1, wherein the method generates a mutation in the target gene that abolishes its expression in the NK cell.

14. (canceled)

15. The method of claim 1, wherein the base editor is BE3, BE4, or ABE8e.

16. The method of claim 1, wherein the NK cell is a primary NK cell.

17. The method of claim 1, wherein the NK cell is stimulated prior to the introduction of editing reagents.

18. A genetically modified NK cell obtained according to the method of claim 1.

19. The genetically modified NK cell of claim 18, wherein the NK cell exhibits increased persistence in a tumor microenvironment relative to an unmodified NK cell.

20. The genetically modified NK cell of claim 18, wherein the NK cell exhibits increased antibody-dependent cellular cytotoxicity relative to an unmodified NK cell.

21. (canceled)

22. A method of treating a disease comprising administering the genetically modified NK cell of claim 18 to a subject in need thereof.

23. (canceled)

24. (canceled)

25. The method of claim 1, wherein the target sequence is one or more target genes, wherein the one or more target genes are selected from aryl hydrocarbon receptor (AHR), cytokine-inducible SH2-containing protein (CISH), killer cell lectin-like receptor subfamily G member 1 (KLRG1), T cell immunoreceptor with Ig and ITIM domains (TIGIT), killer cell lectin-like receptor subfamily C member 1 (KLRC1), programmed cell death protein 1 (PDCD1), CD16A, Fas, or transforming growth factor beta receptor 2 (TGFBR2) and any combinations thereof.

26. (canceled)

27. The method of claim 25, wherein step (b) comprises two or more gRNA for each of the target genes.
